(19) United States
(12) Patent Application Publication
(10) Pub. No.: US 2014/0323573 A1
(43) Pub. Date: Oct. 30, 2014

(54) METABOLIZABLE SALTS AND USE THEREOF IN DIAGNOSTICS AND THERAPY

(71) Applicant: Manfred Rüdinger, (US)

(72) Inventor: Rolf Zander, Mainz (DE)

(21) Appl. No.: 14/363,275

(22) PCT Filed: Nov. 30, 2012
(86) PCT No.: PCT/EP2012/074103

§ 371 Date: Jun. 5, 2014

(30) Foreign Application Priority Data
Dec. 7, 2011 (DE) 10 2011 056 142.0

(51) Int. Cl. A61K 31/131; G01N 33/86

(52) U.S. Cl. CPC A61K 31/131; G01N 33/86
USPC 514/554; 436/69

(57) ABSTRACT

The present invention proposes novel salt compounds for use in the diagnosis or therapy of a patient as well as drugs, medical products, diagnostic compositions or blood products containing a salt compound according to the invention. The salt compounds according to the invention comprise the cation of a metabolisable basic amino acid and the anion of a metabolisable carboxylic acid or an anion of carbonic acid. Those salt compounds therefore have the advantage that they are fully metabolisable and thus do not adversely affect the electrolyte balance or the acid-base balance of the patient. The proposed salt compounds are particularly suitable for the therapy of the liquid and electrolyte balance of a patient but also for adjusting the pH-value in a biological or medical sample, for adjusting the osmolality in infusion solutions, as buffer substances, for feeding biocarbonate to a patient and for inhibiting blood coagulation for example in a patient, in extracorporeal blood treatment in the diagnostic determination of blood coagulation or in the production, storage and preparation of blood products.

The present invention concerns salt compounds for use in diagnosis or therapy of a patient as well as drugs, medicinal products, diagnostic compositions or blood products which contain a salt compound according to the present invention.

In any feed of relatively large amounts of fluid into the body of a patient attention is to be paid to using an electrolyte solution which is as balanced as possible and which has the physiological electrolyte pattern of the plasma with sodium, potassium, calcium and magnesium as well as chloride and the contributions thereof to osmolality as well as a physiological acid-base status with bicarbonate or alternatively other suitable anions. Therefore infusion of a solution balanced in that way cannot cause any therapeutic error—except in respect of volume—. Therefore an object of the present invention is to provide salt compounds which are particularly well suited to the production of electrolyte solutions which are as balanced as possible.

On the other hand there are obviously also forms of fluid therapy, in which operation is implemented other than with physiologically composed solutions in order to correct a state differing from the ideal state or preventively to maintain the physiological conditions in opposition to a circumstance or agent acting in a different direction. What is decisive in the specified cases is that in any administration of solutions containing electrolytes or of solutions which can influence the electrolyte balance, that is effected in such a way that overall it is not just the desired electrolyte balance but also the desired acid-base equilibrium and the ideal osmolality that are achieved, and therefore an object of the present invention is also to provide salt compounds suitable for that purpose.

Theoretically ammonium bicarbonate (also ammonium hydrogen carbonate or NH₄HCO₃) would be suitable for elimination of the two gaseous metabolism end products CO₂ and NH₃ and in addition as a physiological buffer substance in the body fluid balance. Ammonium bicarbonate with its pH-value of 7.4-7.6 (depending on the respective ion strength) has ideal properties as a physiological buffer substance. The pH-value of ammonium bicarbonate results from the pK-values of the two hydrated subunits and the anions resulting therefrom HCO₃⁻ (pK 6.1) and NH₄⁺ (pK 9.0).

In the neutral pH-range of 6 to 8 and in particular in the physiological pH-range of 7.4±0.4 there are scarcely buffer substances which could be therapeutically used. Synthetic buffers which are used in particular in laboratory biology, in the relevant pH-range, are for example Hepes with an almost ideal pK of 7.35 and Tris with a pK of 8.2. Hepes however is not used on the human being. Only Tris—for the therapy of an acidosis—and arginine hydrochloride—for the therapy of an alkalosis—are permitted as medicaments for use on human beings.

Therefore an object of the present invention is to provide salt compounds which are suitable for the therapy of the fluid balance of a patient and in respect of the use of which the electrolyte balance, the acid-base equilibrium and/or the osmolality can be adjusted in the desired fashion in the patient to be treated.

Besides targetedly influencing the fluid balance such as for example in infusion therapy there are a series of therapeutic interventions which have an unwanted influence on the fluid balance. For example in the machine production of blood plasma or blood cell concentrates (plasma- or cytapheresis) relatively large amounts of citrate are frequently added to the blood flow passed extracorporeally through the apparatus for reducing the blood coagulation tendency. The citrate is usually supplied in the form of sodium citrate plus citric acid, for example in the form of ACD-A.

ACD-A (acid citrate dextrose type A) is a solution of citric acid, sodium citrate and D-glucose in water. ACD-A prevents coagulation of the blood, in which the Ca²⁺ ions which are essential for blood coagulation are complexed (bound). ACD-A is traditionally used in the production and storage of blood products for coagulation inhibition.

ACD-A is a strongly hypotonic solution both in vitro (in the laboratory) and also in vivo (in the patient). In diagnostics in vitro ACD-A produces in the blood an acidosis with a BE (base excess; see hereinafter) of −13.8 mmol/l after dilution of 1:10 (9 parts of blood+1 part of solution). When used for blood coagulation inhibition in conjunction with the extracorporeal blood treatment ACD-A can produce a strongly positive BEpot (potential base excess; see hereinafter) in the patient, in accordance with how much of the constituents of the ACD-A solution occur in the patient. In addition ACD-A (Na concentration of Na₃ citrate: of 224.4 mmol/l) in comparison with the blood plasma (Na concentration: 142 mmol/l) is a strongly hypernatriaemic but potassium-free solution (K-concentration of the blood plasma: 4.5 mmol/l).

Although the attempt is made to remove again the sodium citrate introduced into the patient blood by way of the ACD-A solution prior to returning the blood into the body of the patient, it is to be assumed that a not inconsiderable amount of the sodium citrate used remains in the patient blood and can thus lead to impairment of the physiologically normal electrolyte and acid-base status of the patient.

Therefore an object of the present invention is also to provide salt compounds with which blood coagulation can be reduced for example in the therapeutic treatment of a patient by means of a method of extracorporeal blood treatment, without detrimentally influencing the electrolyte and/or the acid-base balance of the patient. In addition those salt compounds are also intended to be capable of serving as an agent for inhibiting blood coagulation for the purposes of targeted therapeutic reduction of blood coagulation in a patient. In addition the salt compounds are also intended to be capable of use in diagnostic determination of blood coagulation or in a diagnostic method in which temporarily uncoagulatable blood is produced, by calcium being complexed (bound) with citrate ions (calcium-free blood) and then involving avoidance of the citrate effect (for example by CaCl₂ addition) with free Ca ions.

According to the invention the object of the present invention is attained in that a salt solution for use in the diagnosis or therapy of a patient is produced, wherein said salt compound is characterised in that it comprises the cation of a metabolisable basic amino acid and either the anion of a metabolisable carboxylic acid or an anion of carbonic acid.

‘Salts’ are compounds comprising an anion and a cation. In an organic salt at least one anion or cation is an organic compound. In the case of the present invention at least the cation, namely the basic amino acid, is an organic compound. In the embodiments of the invention in which the anion is a metabolisable carboxylic acid both the cation and also the anion are organic compounds.

‘Amino acids’ are organic compounds comprising an amino group (—NH₂), a carboxyl group (—COOH), a hydrogen atom and an organic side chain R which are bound to a C atom which is identified as an α carbon atom. Amino acids are of the following general formula:

00

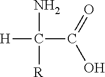

The protonated amino group can react as an acid and the carboxyl group as a base. At a given pH-value the amino acid is present substantially in the form of a zwitterion and is neutral in external behaviour. That pH-value is referred to as the isoelectric point (pH_{IP}).

In ‘basic’ amino acids the side chain R contains basic functional groups, preferably amino groups, which can be easily protonated. The side chains of basic amino acids can react with acid and involve a salt bond, the salt then comprises the singly positively charged amino acid cation and the acid anion.

The basic amino acids have two basic pK_{S}-values and one acid pK_{S}-value. The isoelectric point in basic amino acids is in the basic pH-range between the two basic pK_{S}-values pK_{S2} and pK_{S3} (pH_{IP}>7).

The basic ‘side chain R’ can be a straight- or branched-chain aliphatic, aliphatic-aromatic, aromatic or heterocyclic residue which has at least one basic functional group, preferably an amino group. Preferably the basic side chain R is a straight- or branched-chain aliphatic C₁-C₈ alkyl residue, still more preferably a straight- or branched-chain aliphatic C₁-C₄ alkyl residue.

The ‘cation’ of a basic amino acid is the ion which in total is positively charged externally of the respective basic amino acid. Preferably this involves an ion of the respective basic amino acid, that in total is singly positively charged externally. In an alternative embodiment this involves an ion of the respective basic amino acid, that is in total doubly positively charged externally.

The ‘metabolism’ is the totality of the chemical building-up (anabolism) and breaking-down reactions (catabolism) which occur in living beings in connection with the processes for maintaining life, for example nutrient absorption, energy generation, growth, motion. In connection with the present invention those substances which can be enzymatically metabolised by the body of the patient are referred to as ‘metabolisable’. In other words those substances are substrates which can be chemically converted into other substances by body-specific enzymes of the metabolic system of the patient.

Accordingly the cations of the salt compounds according to the invention are basic amino acids which can be chemically converted into other substances by body-specific enzymes of the metabolic system of the patient.

Preferably the basic amino acids in accordance with the present invention are those amino acids which are intermediate products or end products in the metabolic system of the patient in the healthy state. They can be either proteinogenic or non-proteinogenic amino acids.

‘Non-proteinogenic’ amino acids in accordance with this invention are those basic amino acids which would admittedly occur in the metabolic system of the patient in the healthy state but which are not present as building blocks of the proteins of the patient. Examples of such non-proteinogenic basic amino acids are ornithine and citrulline (both metabolic intermediate products in the urea cycle).

Preferably the non-proteinogenic basic amino acids according to the invention do not have any pharmacological action of their own and they are thus pharmacologically inert.

In another embodiment the basic amino acids are proteinogenic amino acids, wherein the term ‘proteinogenic’ amino acids is used to denote all amino acids which are the building blocks of the proteins of the patient, in which case the proteinogenic amino acids always involve L-amino acids.

The proteinogenic basic amino acids known at the present time include L-lysine, L-arginine and L-histidine. In a preferred embodiment either L-lysine or L-arginine are used as the metabolisable basic amino acid. In an alternative embodiment the D-enantiomers of the proteinogenic amino acids are used as the basic amino acids.

The anion of the salt compound according to the invention is the anion of a metabolisable carboxylic acid or an anion of carbonic acid.

The term ‘metabolisable’ is used in connection with the carboxylic acid of the present invention in the same sense as was defined hereinbefore for the metabolisable amino acid. Accordingly the anions of the salt compounds according to the invention are carboxylic acids which can be chemically converted into other substances by body-specific enzymes of the metabolic system of the patient.

Preferably the carboxylic acid in accordance with the present invention involves those carboxylic acids which are intermediate products or end products in the metabolic system of the patient in the healthy state such as for example lactic acid, gluconic acid and tartaric acid. Further examples of carboxylic acids which are intermediate products or end products in the metabolic system of the patient in the healthy state are the carboxylic acids which are involved in the citrate cycle such as for example acetic acid, citric acid, isocitric acid, α-ketoglutaric acid, succinic acid, fumaric acid, malic acid and oxalic acid.

In an alternative embodiment of the salt compounds according to the invention the anion is an anion of carbonic acid.

Carbonic acid (H₂CO₃) is a two-proton acid whose salts are called carbonates (anion: CO₃²⁻) or hydrogen carbonate (anion: HCO₃⁻). Accordingly the anion of this embodiment can be the carbonate anion or the hydrogen carbonate anion.

The term ‘anion’ of a metabolisable carboxylic acid or an ‘anion’ of carbonic acid is the ion of the respective deprotonated carboxylic acid or carbonic acid, which in total is negatively charged externally. Preferably this involves an ion which in total is singly negatively charged externally (for example: HCO₃⁻) of the respective deprotonated carboxylic or carbonic acid. In an alternative embodiment this involves an ion which in total is doubly negatively charged externally (for example: CO₃²⁻) of the respective deprotonated carboxylic or carbonic acid.

The present invention embraces all combinations of said cations of a metabolisable basic amino acid with the anions of a metabolisable carboxylic acid or the anions of carbonic acid. Examples of those combinations without limitation thereto are: lysine acetate, lysine citrate, lysine isocitrate, lysine-α-ketoglutarate, lysine succinate, lysine fumarate, lysine malate, lysine oxalate, lysine lactate, lysine gluconate, lysine tartrate, aginine acetate, arginine citrate, arginine isocitrate, arginine-α-ketoglutarate, arginine succinate, arginine fumarate, arginine malate, arginine oxalate, arginine lactate, arginine gluconate and arginine tartrate as well as lysine and arginine salts of carbonic acid.

In the case of two-protonic acids in an embodiment of the invention the acid in a deprotonated position can form a salt with the basic amino acid or at both deprotonated positions can form a salt with a basic amino acid. The present invention therefore embraces by way of example and without limitation thereto in particular also the following salt compounds: mono-lysine citrate, mono-lysine isocitrate, mono-lysine-α-ketoglutarate, mono-lysine succinate, mono-lysine fumarate, mono-lysine malate, mono-lysine oxalate, mono-lysine tartrate, mono-arginine citrate, mono-arginine isocitrate, mono-arginine-α-ketoglutarate, mono-arginine succinate, mono-arginine fumarate, mono-arginine malate, mono-arginine oxalate, mono-arginine tartrate and the mono-lysine und mono-arginine salts of carbonic acid as well as di-lysine citrate, di-lysine isocitrate, di-lysine-α-ketoglutarate, di-lysine succinate, di-lysine fumarate, di-lysine malate, di-lysine oxalate, di-lysine tartrate, di-arginine citrate, di-arginine isocitrate, di-arginine-α-ketoglutarate, di-arginine succinate, di-arginine fumarate, di-arginine malate, di-arginine oxalate, di-arginine tartrate and di-lysine und di-arginine salts of carbonic acid.

In the case of two basic amino acids the two amino acids can be identical or can differ from each other. In the case of succinic acid, in an embodiment for example 1 mol of succinic acid can be combined with 1 mol of arginine (Arg succinic) or 1 mol of succinic acid can be combined with 2 mols of arginine (Arg₂ succinate) but also 1 mol of succinic acid with 1 mol of arginine and 1 mol of lysine (Arg Lys succinate). The present invention therefore embraces in particular all combinations of cations of two different metabolisable basic amino acids with the anions of a two-protonic or three-protonic metabolisable carboxylic acid or the anions of the carbonic acid, the listing of which is dispensed with here only because those combinations are in any case already directly clearly and unambiguously disclosed to the man skilled in the art by the information set forth herein.

In the case of three-protonic acids they can correspondingly form in one, two or three positions salt compounds with the same basic amino acid or with different basic amino acids. For example the present invention embraces salt compounds of 1 mol of citric acid and 1 mol of arginine (Arg citrate), salt compounds of 1 mol of citric acid and 2 mols of aginine (Arg₂ citrate) and salt compounds of 1 mol of citric acid and 3 mols of aginine (Arg₃-citrate). The present invention therefore embraces by way of example and without limitation thereto in particular also the following salt compounds: tri-lysine citrate, tri-lysine isocitrate, tri-arginine citrate and tri-arginine isocitrate.

As already mentioned the invention also embraces those salt compounds in which three-protonic acids are combined with different basic amino acids. Thus the invention also embraces for example salt compounds comprising 1 mol of citric acid in combination with 2 mots of aginine and 1 mol of lysine (Arg₂-Lys citrate). The present invention therefore embraces in particular all combinations of cations of three different metabolisable basic amino acids with the anions of a three-protonic metabolisable carboxylic acid, the listing of which is dispensed with here only because those combinations are in any case already directly clearly and unambiguously disclosed to the man skilled in the art by the information set forth herein.

The salt compounds of the present invention have the advantage that they can be completely metabolised, that is to say even upon administration thereof in a large amount into the blood circulation or upon use thereof in a large amount in connection with blood products the above-mentioned disadvantages of the conventional salt compounds used for those purposes do not occur. As the constituents of the salt compounds according to the invention are completely metabolisable they can be readily integrated into the metabolic system of the patient and metabolised by way thereof without any fear of permanently influencing the electrolyte balance and/or the acid-base balance of the patient.

Accordingly the salt compounds according to the invention can advantageously be employed for use in diagnostics or therapy of a patient.

An example of therapeutic use of the salt compounds according to the invention is infusion therapy, by intravenous administration of relatively large amounts of fluid for the purposes of increasing the intravasal fluid volume and/or the extracellular fluid volume in volume replacement, fluid supply and/or electrolyte or osmotherapy.

In a preferred embodiment the salt compound according to the invention can be used as an agent for adjusting the base excess (BE) or the potential base excess (BEpot) in a patient (either acidifying or alkalising).

The ‘BE’ (base excess: [mmol/l]) describes the action that a substance or a mixture of substances has when the substance or the mixture is added to a blood sample in vitro or in vivo. A negative BE-value means that the added substance or mixture causes acidification of the blood. A positive BE-value means that the added substance or mixture leads to alkalisation of the blood. The potential base excess (Bepot) additionally describes the effect in a patient in vivo after the added substance or mixture was potentially metabolised in the patient.

The results shown in Table III of Example No 3 show that Na₃ citrate permits a BE of 0 mmol/l. Na₃ citrate originally therefore does not have any influence on the pH-value or the BE of the blood. It will be noted however that Na₃ citrate has a relatively great influence on the BEpot of the patient and the sodium loading is considerable. In the case of pure citric acid there is no sodium loading on the patient. It will be noted however that citric acid leads to a negative even if reversible BE of the patient as soon as it has been metabolised.

The advantage of the salt compound according to the invention is that the BE of the patient is not influenced by the presence thereof. Preferably the phase deviation (BE) of the patient is 0±10 mmol/l after administration of the substance. In addition the potential base deviation (BEpot) after metabolisation of the substance is preferably only 0±10 mmol/l.

In a further embodiment the salt compound according to the invention is used for adjusting the pH-value in a biological or medicinal sample. In this case too the advantage is that the BE of the sample is not influenced thereby. For example 100 mmol/l of Arg acetate (pK 4.6) has a buffer capacity β of only 7.0 mmol/l/pH. Arg acetate however can buffer towards both sides because β increases towards both sides. If therefore a pH-value of 7.4 is to be set only 7.3 mmol/l/NaOH has to be added.

In still a further embodiment of the invention the salt compound is used as a buffer substance. It is to be noted in this respect that the buffer capacity β of the salt compound is correspondingly greater, the higher the uppermost (most alkaline) pK of the acid is and the more arginine or lysine is present (mono-, di- and tri-amino acid salts). Preferred salt compounds in this connection are therefore those whose uppermost (most alkaline) pK-value>0, still more preferably >12. In addition those salt compounds are preferred, in which more than 1 mol basic amino acid per 1 mol carboxylic acid are combined (for example Arg₂ succinate). Still more preferred are those salt compounds in which more than 2 mols basic amino acid per 1 mol carboxylic acid are combined (for example Arg₃ citrate). Particularly preferred are those salt compounds in which 2 mols (for example Arg₂ succinate) or 3 mols (for example Arg₃ citrate) basic amino acid per 1 mole carboxylic acid are combined.

In a further embodiment of the invention the salt compound is used for adjusting osmolality in infusion solutions. That has the advantage that no inorganic electrolytes which could negatively influence the electrolyte balance of the patient have to be added to the infusion solution. The salt compounds according to the invention act here so-to-speak as ‘osmolalic place holders’, that is to say in an infusion solution the osmolality can be adjusted as desired without an influence on the electrolyte composition.

In still a further embodiment the salt compounds according to the invention are used for the feed of bicarbonate anions in the patient in vivo. The advantage is that here too the addition of possibly detrimental inorganic electrolytes can be dispensed with. In addition there is in that way also the possibility of avoiding the disadvantages of inorganic bicarbonate (CO₂ as volatile base).

In still a further embodiment the salt compounds according to the invention are used for the preparation of bicarbonate in a biological or medical sample.

In another embodiment the salt compounds according to the invention are used for artificial (parenteral) nutrition because as a mixture of amino acid and possibly organic carboxylic acid they represent pH-neutral energy carriers (for example 10 g of Arg acetate provides 35.4 kcal; arginine=4 kcal/g and acetate=3.5 kcal/g).

In an alternative embodiment the salt compound according to the invention serves for use as an agent for inhibiting blood coagulation. For example the salt compound can be used in the therapeutic reduction in blood coagulation in a patient. For that purpose the salt compounds according to the invention can be intravenously administered to the patient in a suitable dosage in the form of an appropriate drug preparation.

In an alternative embodiment the salt compound according to the invention serves for inhibiting blood coagulation in the therapeutic treatment of a patient by means of a method of extracorporeal blood treatment by the salt compound according to the invention being added to the patient blood to be treated. Examples of methods of extracorporeal blood treatment are treatments with a heart-and-lung machine (HLM), extracorporeal membrane oxygenation (ECMO), continuous renal replacement therapy (CRRT), like for example haemofiltration (CVVH), haemodialysis (CVVHD), haemodiafiltration (CVVHDF) and intermittent renal replacement therapy or intermittent haemodialysis (IHD). In those methods the salt compounds according to the invention can be extracorporeally added to the patient blood in an appropriate dosage in the form of a suitable preparation, wherein the advantage of the salt compounds according to the invention is in particular that the salt compounds do not have to be completely removed from the blood again prior to returning the extracorporeally treated blood as the compounds do not have any unwanted influence on the electrolyte and acid-base balance of the patient and can be completely metabolised by the patient metabolic system: non-removed proportions which remain in the patient—in contrast to the conventional citrate—do not alter the BE, the BEpot, the levels of electrolyte concentration or the osmolality of the patient, independently of dilution in the patient blood.

The present invention also embraces the use of the salt compound according to the invention as a diagnostic agent for inhibiting blood coagulation in an in vitro method of blood coagulation diagnostics or in another diagnostic in vitro method in particular when in the method temporarily uncoagulatable blood is produced by calcium being complexed (bound) with citrate ions (calcium-free blood), then involving avoidance of the citrate effect with free Ca ions (for example by CaCl₂ addition). In conjunction with those embodiments the salt compounds according to the invention are added to the sample in a suitable dosage in the form of an appropriate diagnostic preparation.

In the course of a blood coagulation diagnostic process, beginning with taking the blood (pre-analysis), no changes should be made to the pH-value of the sample to be investigated as any pH-change must influence the diagnostic result (Zander: DE 10 2008 022 884, WO 2009/135785 and EP 09 742 003). In principle a pure citrate solution is suitable for that purpose, which because of its 3 pK-values at the blood pH-value of about 7.40 has practically no buffer capacity. Such a solution however has a pH-value of about 8.5. If in contrast solutions with an addition of citric acid are used identified as buffered solutions, the pH-values of the blood sample are markedly altered.

Thus the requirement for a citrate solution should be that the BE of the solution must be 0 mmol/l so that in use of that solution no change in pH-value in the blood-solution mixture occurs. The requirement for a BE of 0 mmol/l can additionally be implemented with bicarbonate (24 mmol/l), but only when there is no citric acid at the same time because bicarbonate remains stable only at the pH-value of 7-9. An embodiment is described hereinafter in Example 5a).

In a further embodiment of the invention the salt compounds according to the invention are used for the inhibition of blood coagulation in the production, storage and processing (for example plasmapheresis and cytapheresis) of blood products. In these embodiments the salt compounds according to the invention are added to the respective blood product in the production, storage and processing thereof in a dosage suitable for inhibiting blood coagulation.

In the aforementioned embodiments in which the salt compound according to the invention is used as an agent for inhibiting blood coagulation the anion of the salt compound according to the invention is the citrate anion in each case.

In a preferred embodiment the salt compound according to the invention is used in the form of a preparation which in comparison with the human blood plasma is isotonic, isonatriaemic, isokaliaemic and/or isohydric. An example of such an embodiment is a citrate-bearing agent for coagulation inhibition, wherein the composition is adapted to the human plasma with arginine citrate: isotonic (osmolality 288±10 mosmol/kg H₂O), isonatriaemic (sodium 142±10 mmol/l), isokaliaemic (potassium 4.5±2 mmol/l), isohydric in vitro (base excess BE 0±10 mmol/l) and in vivo (potential base excess 0±10 mmol/l). Embodiments are described hereinafter in Example 5a) and b). Selectively a solution with an alkalising effect in vivo can also be adopted (potential base excess+5 to +50 mmol/l).

The salt compounds of the present invention can be used in connection with the diagnosis and therapy of all living beings who have a blood circulation system comparable to a human being. Preferably the patient is a mammal. Still more preferably the patient is a human being.

The present invention also embraces preparations which include at least one salt compound according to the invention and in addition at least one pharmaceutical compatible carrier, additive or diluent substance. The carrier, additive or diluent substances which are considered in connection with the aforementioned applications are well-known to the man skilled in the art and their respective suitability for the given purposes of the present invention and the amounts used in that respect depend on the specific problem of the especial state to be treated or diagnosed in the context of the present invention and the overall constitution of the patient.

Preferably the preparations which include at least one salt compound according to the invention involve aqueous solutions of at least one salt compound according to the invention. Such aqueous solutions of the salt compound or compounds according to the invention which are suitable for intravenous administration to a patient are particularly preferred.

In an embodiment the preparation involves a drug preparation for reducing blood coagulation in a patient. In another embodiment the preparation is a preparation or a medicinal product for inhibiting blood coagulation in the therapeutic treatment of a patient by means of an extracorporeal blood treatment method. In still another embodiment the preparation is an additive to a blood product. In a further embodiment the preparation is a preparation or diagnostic composition for inhibiting blood coagulation in blood coagulation diagnostics or in another diagnostic method in which uncoagulatable blood is investigated. In further embodiments the preparation is an infusion solution, a diagnostic or therapeutic buffer solution or a solution for adjusting the pH-value (for example in a biological sample).

The term ‘drug’ in connection with the present invention embraces those substances or preparations of substances which are intended for use in or on the human or animal body and are intended as agents with properties for healing or relieving or for preventing human or animal diseases or diseased complaints or which can be used in or on the human or animal body or administered to a human being or an animal in order either to restore, correct or influence the physiological functions by a pharmacological, immunological or metabolic effect or to produce a medical diagnosis.

The term ‘drug’ in connection with the present invention however does not embrace substances or preparations of substances which fall within the definition of the term ‘medicinal product’.

The term ‘medicinal product’ in connection with the present invention embraces all substances or mixtures of substances which are intended for use on the patient for detecting, preventing, monitoring, treating or alleviating diseases, detecting, monitoring, treating, alleviating or compensating for injuries or disabilities or investigating, replacing or changing a physiological process, and the appropriate main action thereof is achieved in or on the human body neither by pharmacological or immunological means nor metabolically, the action of which however can be assisted by such means or can first be made possible thereby.

‘Diagnostics’, ‘diagnostic agent’ or ‘diagnostic compositions’ are used in accordance with the present invention to denote substances or mixtures of substances which are employed to investigate the state and the function of the organism of a patient. They also serve for disease progress monitoring, therapy monitoring and therapy control. Those substances or mixtures of substances are either used outside the organism on samples taken from the body of the patient or delivered by the body (‘in vitro diagnostics’) or have to be administered to the patient for diagnostic purposes (‘in vivo diagnostics’).

‘Blood products’ in connection with the present invention are blood constituents obtained from patient blood or complete blood which have been processed for transmission (transfusion) to a receiver such as for example erythrocytes concentrate, frozen fresh plasma (FFP), thrombocytes concentrate, granulocytes concentrate, thrombocytes-rich plasma, stem cell preparations and albumin.

For the purposes of the original disclosure it is pointed out that all features as can be seen by a man skilled in the art from the present description and the claims, even if they are described in specific terms only in connection with certain other features, can be combined both individually and also in any combinations with others of the features or groups of features disclosed here insofar as that has not been expressly excluded or technical aspects make such combinations impossible or meaningless. A comprehensive explicit representation of all conceivable combinations of features is dispensed with here only for the sake of brevity and readability of the description.

It is further pointed out that it is self-evident to the man skilled in the art that the embodiments given by way of example in the present application, as set forth for example in the Examples hereinafter, serve only to set forth by way of example the possible embodiments of the invention that are reproduced as configurations by way of example. The man skilled in the art will therefore readily appreciate that in addition all other embodiments having the features or combinations of features according to the invention recited in the claims fall within the scope of protection of the invention. A comprehensive explicit representation of all conceivable embodiments is also dispensed with here only for the sake of brevity and readability of the description.

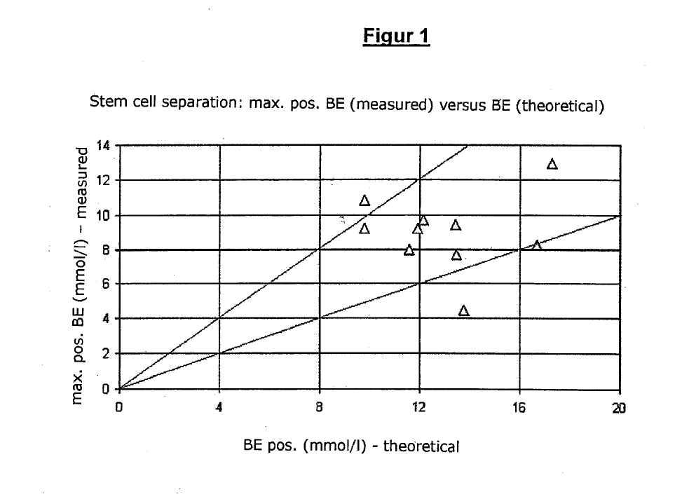

FIG. 1 accompanying this application shows:
a graphic representation of the results of a test for investigating the influence of the ACD-A solution which is usual at the present time on the acid-base balance of patients involving cytapheresis.

### EXAMPLES

### 1. Buffer Properties of the Salt Compounds According to the Invention

Following Table I sets out examples of salt compounds according to the invention, specifying the specific buffer properties thereof.

TABLE I
Metabolisable anionen of basic amino acids;
Acid-base data (37° C.)

| | | | According to calculated data | | | |
|---|---|---|---|---|---|---|
| | | | | NaOH | | |
| | | | | ΔpH | | |
| | | Addition | | 7.2-7.6 | β (at 7.4) | Titr. on 7.4 |
| Material | pK-values | acid mol/l | pH | mmol/ | mmol/l/pH | mmol/ |
| Arginine | 2.0/9.0/12.1 | 0.1 | 10.26 | | | |
| Bicarbonate | carbonic acid | +CO₂ | 7.0 to 7.5 depending on respective | | | |
| | 6.1 | | arginine concentration | | | |
| Acetate | acetic acid | +0.1 | 5.99 | 2.8 | 7.0 | 7.3 |
| | 4.6 | | | | | |
| Lactate | lactic acid | +0.1 | | | | |
| | 3.8 | | | | | |
| Malate | malic acid | +0.2 | 6.67 | 5.4 | 13.5 | 5.8 |
| | 3.5/4.7 | | | | | |
| Tartrate | tartaric acid | +0.2 | | | | |
| | 3.0/4.3 | | | | | |
| Citrate | citric acid | +0.3 | 6.99 | 7.5 | 18.8 | 6.5 |
| | 3.0/4.4/5.8 | | | | | |
| Lysine | 2.2/8.9/10.3 | 0.1 | 9.55 | | | |
| Bicarbonate | carbonic acid | +CO₂ | | | | |
| | 6.1 | | | | | |
| Acetate | acetic acid | +0.1 | 5.99 | 2.5 | 6.3 | 6.8 |
| | 4.6 | | | | | |
| Lactate | lactic acid | +0.1 | | | | |
| | 3.8 | | | | | |
| Malate | malic acid | +0.2 | 6.52 | 4.0 | 10.0 | 4.7 |
| | 3.5/4.7 | | | | | |
| Tartrate | tartaric acid | +0.2 | | | | |
| | 3.0/4.3 | | | | | |
| Citrate | citric acid | +0.3 | 6.88 | 5.9 | 14.8 | 5.8 |
| | 3.0/4.4/5.8 | | | | | |

### Explanation of the Table

1. NaOH mmol/l for ΔpH of 7.2-7.6: how much NaOH in mmol/l is necessary for that ΔpH.

2. β (mmol/l/pH): the buffer capacity calculated therefrom at pH 7.40.

3. Titr on 7.4 (mmol/l): how much NaOH is necessary to set a pH-value of 7.4.

4. Arg₃ citrate was crystallised once, after quantitative addition of H₂O the initial values of the pH and osmolality were restored.

The following conclusions can be drawn from the present data:

1. The buffer capacity β is correspondingly greater, the higher the uppermost (most alkaline) pK of the acid and the more arginine or lysine (mono-, di-, tri-) is present. That permits a targeted selection.

2. If a physiological pH-value of 7.4 is to be set in a 0.1 molar solution then the corresponding additives are specified in the column Titration pH=7.4. It is therefore specified how much NaOH (mmol/l) is to be added to set that pH-value.

3. It is shown for the investigated 0.1 molar solutions that their buffer capacity turns out to be low when for comparison the β-value is specified for normal blood (pH 7.2-7.6) at about 72 mmol/l/pH. That consideration is important when in the case of Arg₃ citrate unbuffered coagulation inhibition is to be produced with a 0.1 molar solution (100 mmol/l), a solution which is usually diluted in the blood at 1:10, which then gives a buffer capacity β of only still 1.9 mmol/l/pH (in comparison with 72 mmol/l/pH).

4. Although metabolically convertible those substances do not have any metabolic effect on the acid-base balance. That will be clear from the following comparisons:
acetic acid (CH₃COOH) acts reversibly as acid (neutral after metabolisation), sodium acetate in contrast acts irreversibly as alkalising;
Arg.HCl (arginine hydrochloride) acts as acid (HCl is non-metabolisable), Arg. HCO₃ in contrast acts neutral, likewise tri-Arg citrate and the like;
ammonium chloride (NH₃+HCl═NH₄Cl) acts as acid (HCl non-metabolisable), ammonium bicarbonate (NH₃+H₂CO₃═NH₄HCO₃) in contrast is neutral in action.

### 2. Osmotic Coefficient

### a) Osmotic Coefficients of Compounds According to the Invention and not According to the Invention

TABLE II

| | Osmotic coefficients (0.1 molar solutions) | | |
|---|---|---|---|
| Substance | Osmot. valence | Osmot. coefficient | |
| Glucose | 1 | 1.013 | (literature) |
| Lysine | 1 | 1.000 | |
| Arginine | 1 | 0.980 | |
| NaCl | 2 | 0.926 | (literature) |
| NaHCO₃ | 2 | 0.926 | (assumed) |
| Arg HCl | 2 | 0.895 | |
| Lys₂ malate | 3 | 0.847 | |
| Arg HCO₃ | 2 | 0.833 | 0.2 m 0.833 100% CO₂ at 37° C. |
| | | | 0.3 m 0.763 100% CO₂ at 37° C. |
| Arg₂ malate | 3 | 0.783 | |
| Lys₃ citrate | 4 | 0.715 | |
| K₃ citrate | 4 | 0.681 | |
| Na₃ citrate | 4 | 0.681 | |
| Arg₃ citrate | 4 | 0.593 | |
| Citric acid | 3 | 0.555 | |

### Examples

A 0.1 molar arginine solution exhibits an osmolality of 98 mosmol/kg H₂O, a 0.1 molar Arg₃ citrate solution has an osmolality of 237.2 mosmol/kg H₂O.

### For Comparison:

Typical solutions used at the present time in in vitro diagnostics and in vivo therapy are:

1. Citrate solution 109 mmol/l, pH-value=about 8.3 (37° C.) (poorly defined as unbuffered), produced from 3.21% (w/v) of tri-sodium citrate dihydrate.

Measurement value: osmolality: 297 mosmol/kg H₂O, pH-value (37° C.)=8.81.

2. Citrate-citric acid solution 109 mmol/l citrate, with 85.5 mmol/l tri-sodium citrate (MW 294.1) plus 23.4 mmol/l citric acid, pH=5.6 (37° C.), produced from 2.515% (w/v) tri-sodium citrate dihydrate plus 0.492% (w/v) citric acid monohydridate (MW 210.1).

Measurement value: osmolality: 272 mosmol/kg H₂O, pH-value (37° C.)=5.42; severely hypotonic solution.

3. ACD-A

| Solution | Citrate | Citr-Sr. | Total citrate | Measurement value osmolality |
|---|---|---|---|---|
| ACD-A | 74.8 | 38.1 | 112.9 | 265 mosmol/kg H₂O |

| | | |
|---|---|---|
| With | 24.5 g/l glucose | 396 mosmol/kg H₂O |

Commentary on the Glucose Present in the Solution:

Although detected in the measurement of a solution the glucose—including in vitro—is osmotically not effective as it equally increases the osmolality both in the plasma and also in the Ery water.

ACD-A is a severely hypotonic solution.

### 3. Influence of Citric Acid and Sodium Citrates on BE, BEpot and the Sodium Concentration

A typical dilution of 1:10 (9 parts of blood+1 part of citrate solution) affords the following effects for the various citrate forms if a typical solution with 100 mmol/l were used for coagulation inhibition:

TABLE III
Effects of various conventional citrate forms on the BEpot

| | | BE | BEpot | Na-addition |
|---|---|---|---|---|
| | | (mmol/l) | (mmol/l) | (mmol/l) |
| 10 mmol/l | citric acid | −30 | 0 | 0 |
| 10 mmol/l | NaH₂ citrate | −20 | +10 | 10 |
| 10 mmol/l | Na₂H citrate | −10 | +20 | 20 |
| 10 mmol/l | Na₃ citrate | 0 | +30 | 30 |

The results in Table III show that Na₃ citrate permits a BE of 0 mmol/l. Na₃ citrate therefore originally exerts no influence on the pH-value or BE of the blood. It will be noted however that Na₃ citrate has a very great influence on the BEpot of the patient and the sodium loading is considerable although at least in vitro that does not play any part, assuming isotony. In the case of pure citric acid the sodium loading of the patient goes. It will be noted however that the citric acid leads to a negative even if reversible BE of the patient as soon as the citric acid has been metabolised.

### 4. Influence of the Salt Compounds not According to the Invention on the Acid-Base Balance

### Example ACD-A

| | | | total- | pH (37° C.) | derived (mmol/l) | |
|---|---|---|---|---|---|---|
| Solution | citrate | Citr-Sr. | citrate | measured | BE | BEpot |
| ACD-A | 74.8 | 38.1 | 112.9 | 4.96 | | |

| | |
|---|---|
| −114.3 (reversible) | −24 |
| | (HCO₃) |

| | total. | −138 | |
|---|---|---|---|
| 224.4 (−24) | total. | | +200 |

A solution which is reversibly strongly acidifying primarily after infusion (BE−138 mmol/l, citric acid), secondarily strongly alkalinising after potential metabolisation (BEpot+200 mmol/l citrate).

Stem cell separation by machine was implemented over about 3 hours using ACD-A. On average all 10 donors received 829 ml of ACD-A which in the extracellular space of 14.8 l (20% of the mean body weight of 74.1 kg) had to produce a reversible, citric acid-induced negative BE of −7.7 mmol/l (138.3 mmol/l×0.829 l/14.8 l) and a potential, citrate-induced positive BE of +11.3 mmol/l (200.4 mmol/l×0.829 l/14.8 l). Because of the time characteristic of the metabolism of citric acid (reversible acidification) and citrate (irreversible alkalisation) however both effects can differ. As however alkalisation predominates it is to be reckoned that this will involve positive BE values. FIG. 1 shows the maximum positive BE values measured in respect of the patients in comparison with the predicted theoretical BE for a 50-100% citrate metabolism. Evidently almost all maximum positive BE values are between the predictions for a 50% and 100% conversion in the patient.

### 5. Example Compositions

a) Use in vitro: total-citrate 97.2 mmol/l (for a later dilution of 1:10)

| | | |
|---|---|---|
| NaHCO₃ | 24.0 | mmol/l |
| K₃citrate | 1.5 | mmol/l |
| Na₃citrate | 39 | mmol/l |
| Arg₃-citrate | 56.7 | mmol/l |

Measurement values: pH (37° C.) 7.25. osmolality 293 mosmol//kg H₂O
Calculated values: buffer capacity (pH 7-8)˜0 mmol/l/pH. BE 0 mmol/l
Properties: isotonisch (288). isokaliaemic (4.5). isonatriaemic (141).
singly isohydric (BE 0 mmol/l)
b) Use in vivo: total-citrate 100 mmol/l

| | | |
|---|---|---|
| NaHCO₃ | 24.0 | mmol/l |
| KCl | 4.5 | mmol/l |
| Arg₃ citrate | 100 | mmol/l |

Measurement values: pH (37° C.) 7.12. osmolality 284 mosmol//kg H₂O
Calculated values: buffer capacity (pH 7-8)˜0 mmol/l/pH. BE 0 mmol/l, BEpot 0 mmol/l
Properties: isotonisch (288). isokaliaemic (4.5). doubly isohydric (BE 0, BEpot 0).

### 5. Example for the Production of Arg₃ Citrate

Firstly citric acid is dissolved in water, then arginine (3-fold molar) is added. After buffering citric acid vs. arginine has been awaited, checking of the pH-value is effected, then addition of citrate or chloride, lastly NaHCO₃ (after buffering) so that there are no free H⁺ ions which could expel HCO₃, then addition of H₂O.

### 6. Use of the Salt Compounds According to the Invention for the Feed of Biocarbonate

If a solution with 48 mmol/l arginine is mixed with 24 mmol/l acetic acid that gives a mixture respectively comprising 24 mmol/l arginine and arginine acetate with a pH-value of about 8.6. That pH-value corresponds to the effective pK value of arginine (ionic strength 160 mmol/l and 37° C.). If that solution is necessarily equilibrated after infusion in the patient to a pCO₂ of about 40 mmHg a maximum of 24 mmol/l arginine HCO₃ will occur. Thus the physiological values of about 24 mmol/l HCO₃ and a pH-value of about 7.4 will occur (for comparison arginine equilibration with pure CO₂ in Table 1).

1-13. (canceled)

14. A preparation for the inhibition of blood coagulation, wherein the preparation is an aqueous solution of at least one salt compound comprising:
a) the cation of the basic amino acid lycine or arginine, and
b) the anion of citric acid,
characterized in that the basic amino acid is selected from the proteinogenic amino acids L-lysine or L-arginine and the aqueous solution in comparison with human blood plasma is isotonic, isonatriaemic, isokaliaemic and isohydric.

15. A preparation according to claim 14, characterized in that the at least one salt compound is selected from mono-lycine citrate, mono-arginine citrate, di-lycine citrate, di-arginine citrate, tri-lycine citrate and tri-arginine citrate.

16. A preparation according to claim 14, characterized in that at least one salt compound is di-arginine-mono-lycine citrate (Arg₂-Lys citrate).

17. A preparation according to claim 14, characterized in that the base excess (BE) of a patient after administration of the preparation is 0±10 mmol/l.

18. A preparation according to claim 14, characterized in that the potential base excess (BEpot) after metabolization of a patient after administration of the preparation is 0±10 mmol/l.

19. A preparation according to claim 14, characterized in that the preparation is used as an agent for inhibiting blood coagulation in the therapeutic reduction in blood coagulation in a patient, in the therapeutic treatment of a patient by means of a method of extracorporeal blood treatment, in diagnostic determination of blood coagulation in vitro or in another diagnostic in vitro method, in which temporarily uncoagulatable blood is produced by calcium being complexed with citrate ions, wherein the anion of the salt compound is respectively citrate.

20. A preparation according to claim 14, characterized in that the use of the preparation for the inhibition of blood coagulation is effected in the therapeutic treatment of a patient by means of an extracorporeal blood treatment method, wherein the extracorporeal blood treatment method is selected from the following: treatment with a heart-and-lung machine (HLM), extracorporeal membrane oxygenation (ECMO), continuous renal replacement therapy (CRRT), haemofiltration (CVVH), haemodialysis (CVVHD), haemodiafiltration (CVVHDF) and intermittent renal replacement therapy or intermittent haemodialysis (IHD).

21. A preparation according to claim 14, characterized in that the preparation is used for the inhibition of blood coagulation in the production, storage and preparation of blood products, wherein the anion of the salt compound is respectively citrate.

22. A preparation according to claim 14, characterized in that the patient is a human being or a mammal.

23. A preparation according to claim 14, characterized in that the preparation is a drug preparation, a medical product, a diagnostic composition for in vitro or in vivo diagnostics or a blood product.

24. A preparation according to claim 14, characterized in that the preparation is an aqueous solution suitable for intravenous administration to a patient.

25. A preparation according to claim 14, characterized in that the preparation additionally includes at least one pharmaceutically compatible carrier, additive or diluent substance.

26. A preparation according to claim 14, characterized in that the at least one salt compound is L-arginine citrate and the preparation is adapted to human plasma with L-arginine citrate as follows: isotonic with an osmolality of 288±10 mosmol/kg H₂O, isonatriaemic with 142±10 mmol/l sodium, isokaliaemic with 4.5±2 mmol/l potassium, isohydric in vitro with a base excess (BE) of 0±10 mmol/l and in vivo with a potential base excess (BEpot) of 0±10 mmol/l.
